(19) United States
(12) Patent Application Publication
(10) Pub. No.: US 2014/0365161 A1
(43) Pub. Date: Dec. 11, 2014

(54) MATERIAL ANALYSIS SYSTEM, METHOD AND DEVICE

(71) Applicant: CSIR, Pretoria (ZA)

(72) Inventors: Thegaran Naidoo, Boksburg (ZA); Johan Hendrik Swart, Pretoria (ZA); Suzanne Hugo, Johannesburg (ZA); Pieter Van Rooyen, San Diego, CA (US)

(21) Appl. No.: 14/362,031

(22) PCT Filed: Nov. 30, 2012
(86) PCT No.: PCT/IB2012/056846

§ 371 Date: May 30, 2014

(30) Foreign Application Priority Data
Dec. 2, 2011 (ZA) 2011/08880

(51) Int. Cl. G01N 21/84; G03H 1/00; G01N 33/49

(52) U.S. Cl. CPC G01N 21/84; G01N 33/49; G03H 1/0005; G03H 2001/005
USPC 702/127

(57) ABSTRACT

The invention relates to a system and method of analysing material as well as to an apparatus for analysing material, particularly, though not necessarily exclusively, biomaterial. The invention entails receiving holographic intensity data comprising at least a holographic intensity pattern associated with a sample of the material of interest and processing, by applying image processing algorithms and techniques, the received holographic intensity data at least to perform one or both steps of detecting and identifying at least one object of interest in the sample thereby at least to generate a suitable output.

### BACKGROUND OF THE INVENTION

THIS INVENTION relates to a material analysis method, system, and device, for example, for analysis of biomaterial such as blood.

Currently, roughly 8 million full blood counts (FBCs) are done per year in South Africa. The FBC is the first and most common pathological test a medical doctor requires when faced with an apparently ill patient. For a FBC in South Africa and elsewhere in the developing world, the blood is drawn from patients in urban and rural clinics, hospitals and doctor's consultation rooms.

For each test, a vial of blood is drawn, temporarily kept in cold storage, and transported by courier via road to the nearest clinical pathology laboratory where the FBC is performed by an automated blood counting machine, whose results are interpreted by a pathologist.

The logistics of this operation contribute significantly to the cost of the test. The results of by far the majority of the tests are printed, interpreted at the laboratory by the pathologists and then communicated and sent back to the requesting physician or clinic, whom then treats the patient. The typical turnaround time is 48 hours.

It will be noted that in some cases, digital holographic microscopes may be used by laboratories to facilitate testing. However, these devices are cumbersome and may require specialist operators to operate the same.

Accordingly, it is an objective of the present invention to address or at least ameliorate the abovementioned problems and/or disadvantages; or to provide an alternative for conventional systems, devices and methods.

### SUMMARY OF THE INVENTION

According to a first aspect of the invention there is provided a method of analysing material, the method comprising:
receiving holographic intensity data comprising at least a holographic intensity pattern associated with a sample of a material of interest, the holographic intensity data being captured by a data capturing means; and
processing the received holographic intensity data at least to perform one or both steps of detecting and identifying at least one object of interest in the sample.

The step of processing the received holographic intensity data may comprise at least the steps of:
determining one or more data key-points from the received holographic intensity data, the holographic intensity data being associated with a discrete location in a propagation space comprising a three-dimensional space over which illumination, associated with the data capturing means, propagates to facilitate capturing of the holographic intensity data; and
comparing the determined data key-points to at least one pre-determined object descriptor associated with an object to determine a match thereby facilitating one or both steps of detecting and identifying at least one object of interest in the sample, wherein the object descriptor is propagation space invariant.

The method may comprise providing a plurality of object descriptors, each object descriptor may comprise a plurality of descriptor subsets associated with a plurality of desired discrete locations in the propagation space respectively, wherein each descriptor subset may comprise one or more descriptor key-points.

The method may comprise the prior steps of determining the object descriptors, which steps may comprise, for each object:
receiving an image of the object;
applying a waveform propagation algorithm to the received image for a plurality of discrete locations across the propagation space thereby to generate a plurality of holographic intensity patterns corresponding to the discrete locations across the propagation space;
determining descriptor key-points for each generated holographic intensity pattern across the propagation space; and
using the determined descriptor key-points and information indicative of the associated discrete locations across the propagation space to generate the object descriptor associated with the object.

It will be noted that the plurality of generated holographic intensity patterns may be artificially generated by the waveform propagation equation. Though the method comprises automatically generating artificial holograms to train on, it will be appreciated that in some example embodiments, the method may comprise determining descriptor key-points for object descriptor determination by generating a plurality of physical holograms manually to train on.

The image of the object typically comprises a microscope image of the object.

The method may further comprise:
generating object descriptor subsets by associating the determined descriptor key-points and the corresponding discrete location in the propagation space;
generating the object descriptor associated with the object by associating each generated descriptor subset corresponding to the object; and
storing the generated object descriptor in the database.

In one example embodiment, the object descriptor is additionally scale space invariant, the method may therefore comprise:
generating a scale space for each of the plurality of holographic intensity patterns generated across the propagation space by applying a blurring algorithm to each of the generated holographic intensity patterns thereby generating blurred images;
determining differences between the generated blurred images by subtracting the same from each other;
locating external scale invariant key-points in the determined differences; and
using the scale invariant key-points to generate the scale space invariant object descriptor.

It will be noted that the method may comprise determining the accuracy of the match by:
applying a reconstruction algorithm to the received holographic intensity data to reconstruct the received holographic intensity data back to the discrete location in the propagation space associated with matching key-points;
deriving key-points at this location in the propagation space;
comparing the newly derived key-points to the object descriptor to determine confidence in a match.

The method may comprise receiving holographic intensity data in either a hardwired fashion from the data capturing means or wirelessly from a plurality of geographically distributed analysis stations each comprising data capturing means.

The method may comprise controlling the data capturing means to generate holographic data comprising at least a holographic intensity pattern associated with the sample.

The method may comprise;
generating output data associated with one or both of the detection and identification operations; and
transmitting the output data via hardwired or wireless data means to a user interface module at least for output thereby.

The method may comprise:
classifying detected or identified objects of interest by determining a sum of similar objects of interest;
generating an image of the sample by reconstructing the received holographic intensity data;
generating output data comprising one or both of the determined sum and the generated image of the sample; and
transmitting the output data via hardwired or wireless data means to a user interface module for output thereby.

According to a second aspect of the invention there is provided a material analysis system comprising:
a memory device storing data;
a data receiver module being in data communication with a data capturing means and configured to receive holographic intensity data comprising at least a holographic intensity pattern associated with the sample of the material of interest captured by a data capturing means; and
an image processor configured to process the received holographic intensity data at least to perform one or both operations of detecting and identifying at least one object of interest in the sample.

The image processor may comprise:
a key-point extraction module configured to determine one or more data key-points from the received holographic intensity data, the holographic intensity data being associated with a discrete location in a propagation space comprising the space over which illumination, associated with the data capturing means, propagates to facilitate capturing of the holographic intensity data; and
an object classifier configured to compare the determined data key-points to at least one pre-determined object descriptor, stored in the memory device, associated with an object to determine a match thereby facilitating one or both steps of detecting and identifying at least one object of interest in the sample, wherein the object descriptor is propagation space invariant.

The memory device may store a plurality of object descriptors, each object descriptor may comprise a plurality of descriptor subsets associated with a plurality of desired discrete locations in the propagation space respectively, wherein each descriptor subset may comprise one or more descriptor key-points.

The material analysis system may comprise a training module configured to determine the object descriptors, wherein the training module is configured, for each object, to:
receive an image of the object;
apply a waveform propagation algorithm to the received image for a plurality of discrete locations across the propagation space thereby to generate a plurality of holographic intensity patterns corresponding to the discrete locations across the propagation space;
determine descriptor key-points for each generated holographic intensity pattern across the propagation space; and
use the determined descriptor key-points and information indicative of the associated discrete locations across the propagation space to generate the object descriptor associated with the object.

The data receiver module may be in either hardwired data communication with the data capturing means or in wireless data communication a plurality of geographically distributed analysis stations each comprising data capturing means.

The system may comprise the data capturing means or a plurality of geographically distributed analysis stations each may comprise the data capturing means, wherein each data capturing means may comprise a digital holographic microscope arrangement which may comprise at least an illumination source configured to generate illumination and an image sensor configured to generate holographic intensity data in response to the generated illumination incident thereon, in use, wherein the propagation space may comprise at least part of the three-dimensional space between the illumination source and the image forming means.

The digital holographic microscope arrangement may further comprise:
a spatial filter located at a predetermined distance from the illumination source, the spatial filter comprising at least one illumination aperture for passage of illumination from the illumination source therethrough; and
a sample holder removably locatable at a predetermined distance from the spatial filter, the sample holder being configured to hold the sample of material of interest, wherein the image sensor is spaced from the sample holder such that, in use, illumination from the illumination source propagates from the illumination source through the illumination aperture, through the sample holder holding the sample of the material of interest, and onto the image sensor which, in response to the illumination incident thereon, generates the holographic intensity data of the sample of the material of interest; wherein the propagation space comprises the three-dimensional space over which illumination from the illumination source, or propagating from one or both of the illumination aperture and sample holder, propagates to reach the image sensor thereby to form the holographic intensity data.

The system may comprise a user interface module configured to receive user inputs and output, and store in the memory device, at least generated output data associated with the one or both of the operations of detection and identification by the image processor module.

The system may be biomaterial analysis system for analysing a sample of biomaterial associated with a human user, the system may therefore comprise a user interaction module configured to generate a user profile for at least one user of the system in the memory device, the user profile storing generated output data associated with a particular user.

According to a third aspect of the invention, there is provided a material analysis device comprising:
a housing configured removably to receive an sample holder carrying a sample of a material of interest, in use;
a data capturing means locating in the housing for capturing a holographic intensity pattern of the sample of the material of interest;
a memory device storing data;
an image processor configured to process the captured holographic intensity data at least to perform one or both operations of detecting and identifying at least one object of interest in the sample thereby to generate output data associated with said operations; and
a user interface configured to receive user input and to output information comprising at least output data generated by the image processor.

The image processor may comprise:
a key-point extraction module configured to determine one or more data key-points from the received holographic intensity data, the holographic intensity data being associated with a discrete location in a propagation space comprising the space over which illumination, associated with the data capturing means, propagates to facilitate capturing of the holographic intensity data; and
an object classifier configured to compare the determined data key-points to at least one pre-determined object descriptor, stored in the memory device, associated with an object to determine a match thereby facilitating one or both steps of detecting and identifying at least one object of interest in the sample, wherein the object descriptor is propagation space invariant and comprises a plurality of descriptor subsets associated with a plurality of desired discrete locations in the propagation space respectively, and wherein each descriptor subset comprises one or more descriptor key-points.

The data capture means may comprise a digital holographic microscope arrangement which may comprise:
an illumination source configured to generate illumination;
a spatial filter located at a predetermined distance from the illumination source, the spatial filter comprising at least one illumination aperture for passage of illumination from the illumination source therethrough; wherein the sample holder is removably locatable at a predetermined distance from the spatial filter; and; and
an image sensor spaced from the sample holder, the image sensor being configured to generate at least a digital holographic intensity pattern of the material of interest in the sample holder in response to generated illumination incident thereon, in use, wherein the propagation space comprises the space over which illumination from illumination source, or propagating from one or both of the illumination aperture and sample holder propagates, to reach the image sensor thereby to form the holographic intensity data.

The device may comprise a communication module configured to receive data and transmit data wirelessly from the device.

The device may be a biomaterial analysis device for analysing a sample of biomaterial associated with a human user, the device therefore comprising a user interaction module configured to generate a user profile for at least one user of the device in the memory device, the user profile storing generated output data associated with a particular user of the device.

According to a fourth aspect of the invention, there is provided a non-transitory computer readable storage medium comprising a set of instructions, which when executed by a computing device causes the same to perform a method as described above.

### BRIEF DESCRIPTION OF THE DRAWINGS

FIG. 1 shows a schematic diagram of a material analysis system in accordance with an example embodiment of the invention;

FIG. 2 shows a front perspective view of an analysis station of FIG. 1 in more detail with the sample holder in the first position;

FIG. 3 shows a rear perspective view of an analysis station of FIG. 1 in more detail with the sample holder in the first position;

FIG. 4 shows a front perspective view of an analysis station of FIG. 1 in more detail with the sample holder in the second position;

FIG. 5 shows at least a portion of the schematic diagram of the material analysis system in more detail in accordance with an example embodiment of the invention;

FIG. 6 shows a schematic sectional view through the analysis station in accordance with the invention illustrating the data capture means in accordance with the invention in more detail;

FIG. 7 shows a schematic diagram of a material analysis device in accordance with an example embodiment of the invention illustrating the functional modules associated with the device;

FIG. 8 (a) shows an example original conventional bright field microscope image of a sample of a material, a USAF test slide;

(b) shows an image of a generated holographic intensity pattern of (a);

(c) shows a reconstructed image of the holographic intensity pattern of (b);

FIG. 9 (a) shows a hologram of blood smear obtained from a digital in-line holography microscope system;

(b) shows a reconstructed image of blood smear, and

(c) shows a comparison to image of blood smear obtained using conventional bright field microscope (400×);

FIG. 10 (a) shows a bright field microscope image of blood smear sample;

(b) shows a corresponding hologram to (a);

(c) shows a reconstructed image corresponding to (b) for red blood cells to be in focus;

(d) shows a reconstructed image corresponding to (b) for white blood cells to be in focus;

FIG. 11 (a) shows an example of bright field microscope image of blood smear with 1 white blood cell;

(b) shows annotated image of (a) for red blood cells;

(c) shows a location image for red blood cells;

(d) shows a location image for white blood cells;

(e) shows an image illustrating a single white blood cell in the sample;

FIG. 12 shows a high level flow diagram of a method in accordance with an example embodiment;

FIG. 13 shows another high level flow diagram of a method in accordance with an example embodiment; and

FIG. 14 shows a diagrammatic representation of a machine in the example form of a computer system in which a set of instructions for causing the machine to perform any one or more of the methodologies discussed herein, may be executed.

### DESCRIPTION OF PREFERRED EMBODIMENTS

In the following description, for purposes of explanation, numerous specific details are set forth in order to provide a thorough understanding of an embodiment of the present disclosure. It will be evident, however, to one skilled in the art that the present disclosure may be practiced without these specific details.

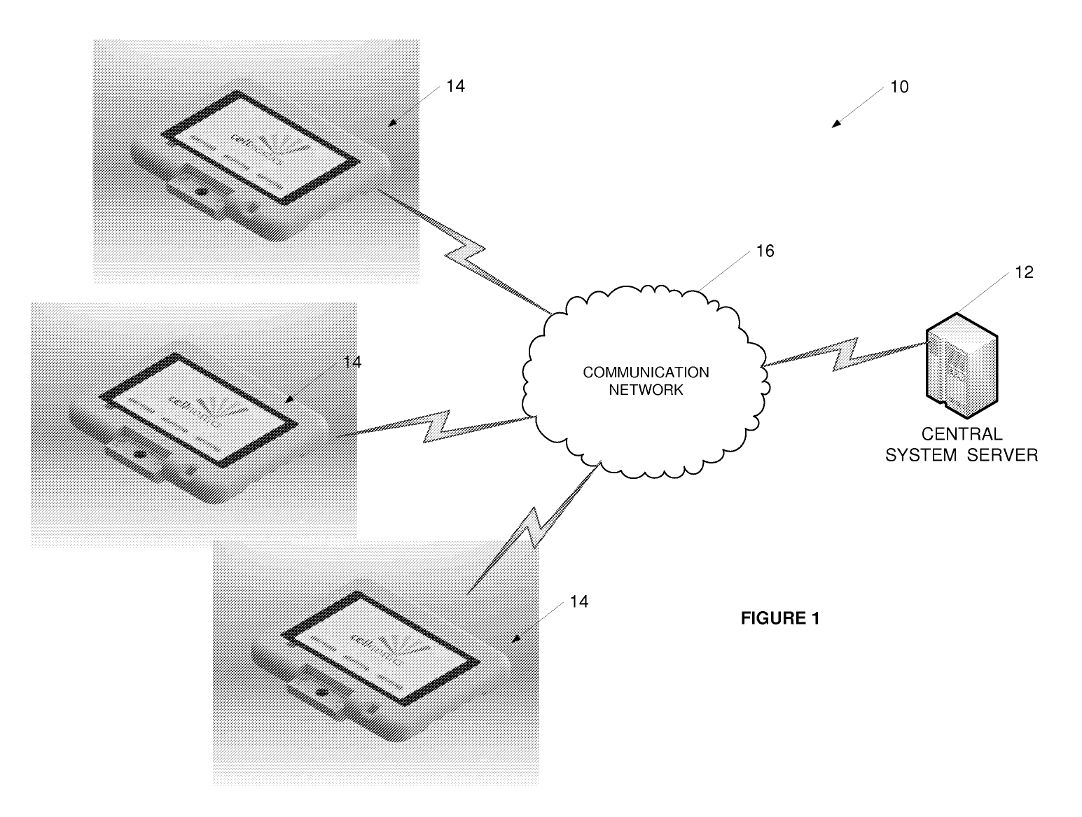

Referring to FIG. 1 of the drawings, a system in accordance with an example embodiment of the invention is generally indicated by reference numeral 10. The system 10 is typically a material analysis system for analysing material, biological or non-biological, and particularly objects of a microscopic scale with fine detail. Though the invention disclosed herein may find application in analysis of any material of interest, the example embodiments will be described with reference to a preferred example embodiment whereby the system is a biomaterial analysis system 10. Biomaterial may comprise any biological material of interest associated with plant or animal life. In the example embodiment present system 10 under discussion, the biomaterial is associated with a human and may comprise blood, tissue, or the like.

For brevity, it will be noted that materials being investigated and analysed by the system are referred to as materials of interest. The system 10 is configured to analyse a sample of a material of interest to detect or identify one or more objects of interest therein. In the example where the material of interest is human blood, the blood cells (red or white) may be objects of interest, the white and red blood cells being different types of objects.

The system 10 may comprise a central system server 12 in wireless data communication with a plurality of geographically spaced or distributed data capture stations 14 via a communications network 16. The communications network 16 may be a radio frequency or mobile telecommunications network, for example, Wi-Fi network or a GSM (Global System for Mobile Telecommunications) network. The communications network 16 may be a packet-switched network and may form part of the Internet. Instead, the communications network 16 may be a circuit switched network, public switched data network, or the like.

The server 12 need not necessarily comprise a single server at one location, but may be distributed across a plurality of distributed networked servers displaced at geographically spaced locations in data communication with each other via, for example, communication network 16. However, a single server is illustrated for ease of explanation. Similarly, though the system 10 may comprise a plurality of stations 14, only three are illustrated.

Each data capture station 14 is typically located at a remote location which is usually inaccessible to proper healthcare facilities, etc. The system 10 thus conveniently provides a point of care system for use in remote locations as the wireless functionality assists in this regard.

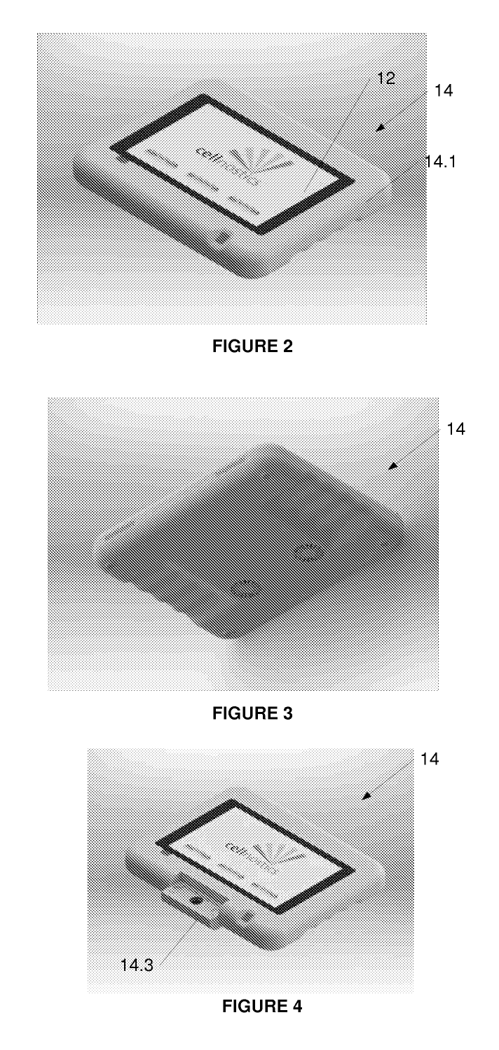

In this regard, turning to FIGS. 2 to 4, where the station 14 is illustrated more clearly. The station 14 conveniently comprises a rugged housing 14.1 constructed from a durable material to withstand use in remote un-urbanised areas. To facilitate ease of use, the housing 14.1 is planar tablet-like having two opposed major faces. A user interface 29 (FIG. 2) may be provided in the housing 14.1, the user interface 29 comprising at least a touch responsive screen 14.2 disposed on one major face of the housing 14.1. The screen 14.2 may display information and a GUI (associated with the interface 29) and may correspondingly receive touch inputs from a user in order at least to control the station 14. These may be done in any conventional manner.

The station 14 is portable and therefore is relatively lightweight and comprises gripping formations to allow for ease of use of the station 14. The station may have the following dimensions: 315 mm×250 mm with a height of 45 mm in an example embodiment.

The housing 14.1 is also configured to removably receive a sample holder carrying a sample of a material of interest, for example, blood (described below) in an illumination sealed manner preventing ambient light from entering the housing 14.1. In one example embodiment, the housing 14 comprises a flap rotatable 14.3 rotatable between a first position in which the flap is exposed for location or removable of the sample holder from the flap 14.3 and a second position whereby the flap 14.3 rotatably closes to introduce the sample holder into the housing 14.1 in a illumination sealed manner.

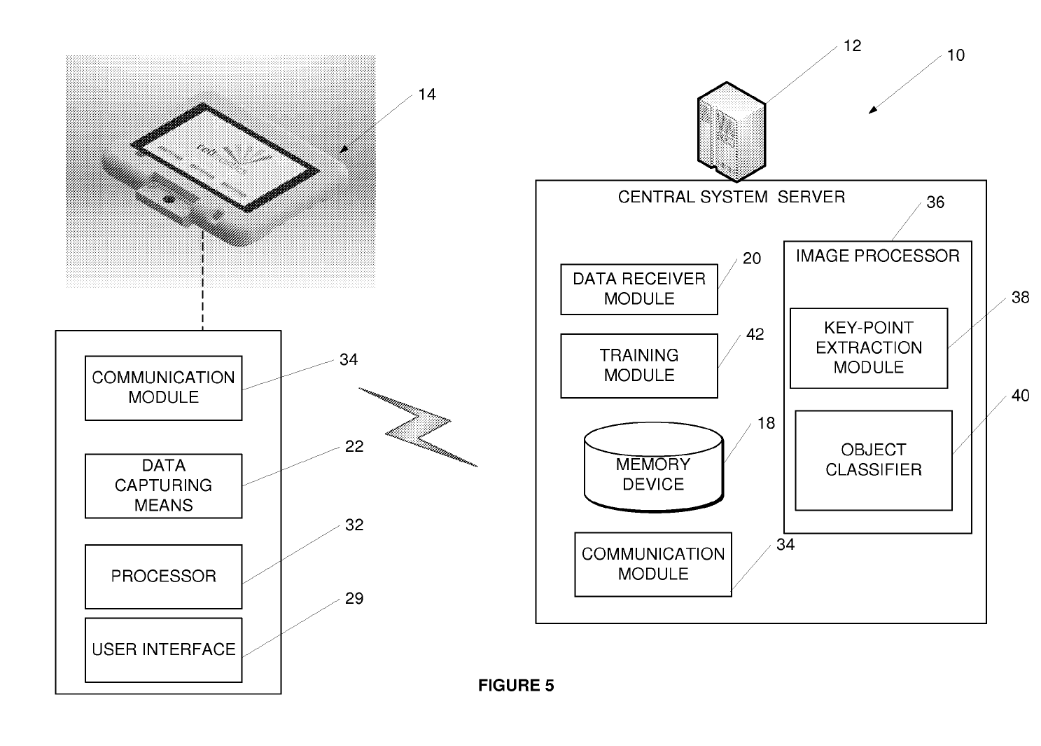

Referring now to FIG. 5 of the drawings where a more detailed illustration of the system 10 as illustrated in FIG. 1 is provided. A single instance of the station 14 and the server 12 is illustrated for ease of illustration. The system 10, particularly the central system server 10, comprises a database or memory device 18 storing non-transitory data. The database 18 may be one or more suitable devices located at one or more locations but in data communication with each other to provide a means for storage of information digitally.

It will be noted that the server 12 may be a computer operated and may comprise one or more processors having non-transitory computer readable medium/s, for example, the database 18 storing instructions or software which directs operation of the server 12 as herein described. Steps described with reference to method disclosed herein are typically achieved by application of one or more processing steps associated with the description as described herein.

In any event, the system 10, particularly the server 12 and the stations 14, comprises a plurality of components or modules which correspond to the functional tasks to be performed by the system 10. The components, modules and means described in the context of the specification will be understood to include an identifiable portion of code, computational or executable instructions, data, or computational object to achieve a particular function, operation, processing, or procedure. It follows that these components, means or modules need not be implemented in software; but may be implemented in software, hardware, or a combination of software and hardware. Further, these components, means or modules need not necessarily be consolidated into one device, particularly in the case of the server 12, but may be spread across a plurality of devices.

The server 12 comprises a data receiver module 20 being in data communication with a data capturing means 22 of the station 14, the data receiver module 20 being configured to receive holographic intensity data comprising at least a holographic intensity pattern or image associated with a sample of the material of interest captured by a data capturing means 22.

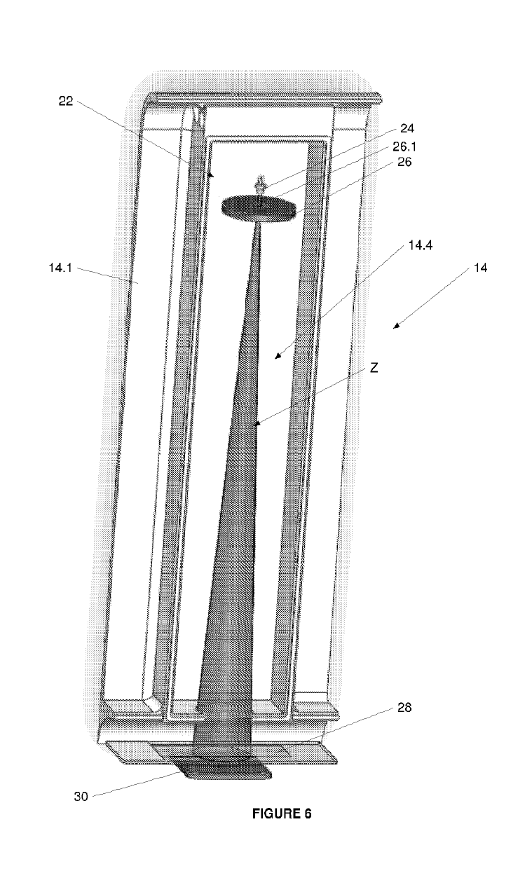

Referring additionally to FIG. 6 of the drawings, wherein the data capturing means 22 is generally illustrated within the housing 14.1. The data capturing means 22 typically comprises a digital holographic microscope arrangement disposed in a light insulated chamber 14.4 defined in the housing 14.1. Though the illustrated embodiment approximates an in-line digital holographic microscopy arrangement, it will be appreciated that off-axis approaches may be used as well. It follows that the digital holography microscope arrangement provided allows for the use of fundamental principles of holography including propagation and interference of light waves, which can be explained using scalar diffraction theory.

The holographic microscope arrangement comprises an illumination source 24 configured to generate illumination. The illumination source 24 comprises an LED (light emitting diode) light source, for example, an infrared laser diode (808 nm) or a blue laser diode (408 nm). A planar spatial filter 26 is located at a predetermined distance from the illumination source 24, the spatial filter 26 comprising at least one circular illumination aperture 26.1 of approximately 50 μm diameter for passage of illumination from the illumination source 24 therethrough. The shape and/or dimension of the illumination aperture 26.1 are selected advantageously to improve the collimation of the light or illumination from the illumination source 24. In other words, it will be noted that the function of the aperture 26.1 is to create a collimated beam before the light waves interact with the sample of material. It follows that this may be accomplished in ways other than that described in the present example embodiment.

In any event, the filter 26 is disposed transverse to a direction of propagation of illumination from the illumination source 24. Illumination emitted from the aperture 26.1 typically comprises diffracted light waves which propagate over a propagation space Z. The propagation space Z may be the defined loosely as the space over which light from the means 18 or diffracted light from the filter 20 propagates to facilitate generating the hologram. The propagation space Z may be a space, for example, a three-dimensional physical space. However, for the present description, the propagation space Z may correspond to single dimension parallel to the main axis of propagation of illumination or light waves from the illumination source 18 and this could be parameterised by Z.

The propagation space Z may be uniquely associated with the data capture means 22. It follows that in industrially replicable stations 14, the propagation space Z is selected to be desirably similar across similar stations.

In any event, the means 22 is configured to receive a sample holder or insert 28 holding a sample of the material of interest, in a removable fashion as described above, at a predetermined distance from the spatial filter 26 and hence the illumination source 24. It follows that the flap 14.3, is configured to receive the sample holder 28 in the first position and bring the same into a predetermined and desired position relative to the means 22, in use, in the second position. In this way, accuracy of the system 10 is further enhanced.

The sample holder 28 is configured to hold a sample of material in the propagation space Z of the illumination from the illumination aperture 26.1. The material in the sample holder 28 typically comprises objects of interest 19, for example, blood cells. The sample holder 22 may therefore comprise a transparent planar microscope slide 28, constructed of glass. The slide 28 may be a conventional slide used in microscopic applications.

The means 22 lastly comprises an image sensor or image recording means 30 located at a predetermined distance from the sample holder 28 in the propagation space Z of the illumination from the sample holder 28. The image sensor 30 is typically configured to generate at least the digital holographic intensity pattern of the material in the sample holder 28 in response to the illumination incident thereon from the source 24 across the propagation space Z. In this way, the means 22 effectively captures the holographic intensity pattern or image of the sample.

The image sensor 30 may be selected from a charge coupled device (CCD) or preferably a complementary metal oxide semiconductor (CMOS) image sensor which is disposed substantially transverse to the illumination propagation space Z. The image sensor 30 may be a 1/2.5-Inch 5MP CMOS digital image sensor 30 with a 2.2 μm×2.2 μm pixel size.

It must be noted that the propagation space Z preferably comprises the space, for example, the entire three-dimensional space, or Z-axis in some example embodiments, over which illumination or light waves from the illumination source 24 or diffracted light from the filter 26 propagates, through the sample holder 28, to reach the image sensor 30 thereby facilitating generation of holographic intensity data.

The slide 28 may be receivable on a tray associated with the flap 14.3 such that operation of the flap 14.3 to the second position introduces the slide 28 into the chamber 14.4 in a light insulated manner to be disposed operatively adjacent the sensor 30 in the propagation space Z. The tray may be shaped and dimensioned to receive the slide 28. In this regard, the slide 28 may have the following dimensions 76 mm×26 mm×1 mm. In addition, it will be noted that the material in the slide 28 may be stained in the case of, for example, blood in a similar manner as a pathologist usually would for analysing the same.

The means 22 is typically lens-less and the digital holographic intensity data comprising holographic intensity patterns generated by the CMOS image sensor 30 may comprise a matrix of pixels having pixel values corresponding to parameters such as pixel intensity, etc. associated with the holographic intensity data. In some example embodiments, the pixel values may be calculated from the values of one or more adjacent pixels for the purpose of image enhancement. It will be noted that to better estimate a pixel value, one could use information from adjacent pixels. Further accuracy could be achieved with super-resolution techniques, which in this case could be based on varying (independently or together) phase, wavelength, and relative spatial displacements between illumination source 24 and sensor or image sensor 30.

The housing 14 may be shaped and dimensioned to provide the chamber 14.4 as well as provide means to locate each component of at least the means 22 and the slide 28 in a secure manner therein at specific pre-determined locations. This advantageously ensures that tolerances between sensitive components are maintained thereby facilitating accuracy of operation of the station 14, in use, especially in rural areas where the rugged construction of the station 14 is important.

In one example embodiment, not necessarily the preferred example embodiment, the distance between the aperture 26.1 and the sample holder 28 is approximately 200 mm to ensure a planar wave at the object plane. The distance between the sample holder 28 and the image sensor 30 may be 2 mm. It will be appreciated that these dimensions may be varied depending on factors such as the dimensions of the station 14, etc.

Returning to FIG. 5 of the drawings, the station 14 also comprises a processor 32 for directing operation of the station 14. To this end, the station 14 may include a machine-readable medium, e.g. memory in the processor 32, main memory, and/or hard disk drive, which carries a set of instructions to direct the operation of the processor 32. It is to be understood that the processor 32 may be one or more microprocessors, controllers, or any other suitable computing device, resource, hardware, software, or embedded logic.

In addition, the station 14 comprises a communication module 34 to facilitate wireless communication with the central system server 12 via the communications network 16. The system server 34 may comprise a suitably matched communication module 34 to facilitate communication via the network 16 and therefore the same reference numeral will be used to indicate the same. The communication modules 34 may comprise one or more modem, antenna, or the like devices to facilitate wireless communication via the network 16 in a wireless fashion. In the illustrated example embodiment, the module 34 facilitates data coupling or communication between the receiver module 20 and the station 40 in a wireless fashion. The station 14 is therefore configured to transmit holographic intensity data captured by the data capturing means 22 wirelessly to the central system server 12 for processing thereby.

It follows that the server 12 therefore comprises an image processor 36 configured to process the holographic intensity data received from the station 14, via the module 20 at least to perform one or both operations of detecting and identifying at least one object of interest in the sample received by the station 14.

The steps of detecting and identifying advantageously provide a more robust analysis approach in an automated manner as compared to many existing systems which merely provides reconstructed holograms for analysis by healthcare professionals.

To further enhance processing of the data received, the image processor 36 comprises modules, which may be modules as hereinbefore defined. In particular, the image processor 36 comprises a key-point extraction module 38 configured to determine one or more data key-points from the received holographic intensity data, the holographic intensity data being associated with a discrete location in the propagation space Z associated with the data capturing means 22 as hereinbefore described. In one example embodiment, the module 38 traverses the pixels of the received holographic intensity image and selects pixels with intensity values of interest, for example, location of local maxima and minima positions, etc, in a conventional manner. It will be noted that the data key-points determined correspond to one or more pixels of interest as selected by the module 38. In some example embodiments, external points may also extracted from the difference of two adjacent snapshots through scale space. This may reduce the number of key points detected to more salient ones.

The image processor 36 further comprises an object classifier 40 configured to compare the determined data key-points to at least one pre-determined object descriptor, stored in the memory device 18, associated with an object to determine a match thereby facilitating one or both steps of detecting and identifying at least one object of interest in the sample, wherein the object descriptor is propagation space invariant. Taking blood cells as objects, each type of blood cell (red and white) may comprise a particular identifier associated therewith which is propagation space invariant by comprising a plurality of descriptor subsets, wherein each descriptor subset comprises a plurality of descriptor key-points and information indicative of an associated discrete location in the propagation space Z.

As will be described, key-points may be collected over the propagation space Z and are therefore localized to the propagation space Z. The collection of key-points may form an object descriptor for the object of interest. It follows that the object descriptor may become propagation space invariant to allow detection and/or identification of an object of interest in a propagation space invariant manner, while the subset of key-points that lead to detection may additionally allow the localization of the object of interest in the propagation space Z.

For example, a red blood cell descriptor will have descriptor key-points [X, Y, Z] at discrete location 1 in the propagation space Z, and [A, B, C] at discrete location 2 in the propagation space Z. An extracted data key-point matching [X, Y, Z] will enable the object classifier 40 to determine that the object in the sample of material (blood sample) is a red blood cell, which is in turn provided at location 1 in the propagation space. In this way, objects in a volume are identified and located in a computationally efficient manner.

It follows that the object descriptor may become propagation space invariant to allow detection and/or identification of an object of interest in a propagation space invariant manner, while the descriptor subset of key-points that lead to detection may additionally allow the localization of the object of interest in the propagation space Z.

The image processor 36 is typically configured to generate output data associated with the detected or identified objects of interest. For example, the image processor 36 may count the number of occurrences of a detected or identified object which in the case of blood will be a blood count (red or white). The server 12 may be configured to transmit the generated output data to the station 14 via the communication modules 34 for display via the display 14.2 of the user interface 29. A user may, by way of the user interface 29, generate instructions to be transmitted to the server 12 to instruct the server to transmit one or more specific items of data for display thereby.

The image processor 36 is further configured to apply a reconstruction algorithm to the received hologram thereby to produce a reconstructed image of the hologram received. The reconstructed image may form part of the output data transmitted to the station 14. The image processor 36 may be configured to pre and post process images to improve quality of the reconstructed images and refine the quality thereof. In this regard, the module 36 may be configured to perform image enhancement by applying an additive high-pass filter.

To improve the resolution of the reconstructed images further, techniques such as super-resolution could be implemented. Super resolution could be achieved by using multiple sources or enabling multiple view points of the object or by placing the object in multiple positions. Super resolution could also be achieved by observing the object at multiple frequencies or at multiple phases. Any one or combination of these techniques could be used.

The invention advantageously assists, at least health care professionals, in remote locations. For example, a doctor in a remote location with access only to a station 14 may select, via the user interface 29, to receive an image of a sample of blood as well as a white blood cell count associated with a sample of blood taken, the image processor 36 counts the detected or identified white blood cells in a conventional manner, reconstructs the received hologram to generate a reconstructed image and transmits the same to the station 14 for viewing by the doctor via the display 14.2 associated with the station 14. In this way, a doctor may advantageously be empowered to provide healthcare assistance in the most remote of locations.

It will be noted that the object descriptors are important to the invention. In this regard, in order to determine the object descriptors for each object of interest, the server 12 advantageously comprises a training module 42 for generating the object descriptors for use by the image processor 36 in a manner as hereinbefore described. It will be understood that the object descriptors need not be generated by the server 12 and may be generated externally and merely used by the server 12.

In any event, the module 42 is configured to receive an image of the object. In this case, the image received by the module 42 is a conventional microscope image and not a hologram. However, in some example embodiments, the module 42 receives a hologram which may be reconstructed for use in a similar manner as the conventional images.

The module 42 is further configured to apply a waveform propagation algorithm to the image received to generate a plurality of holographic intensity patterns corresponding to different discrete locations across the propagation space Z. In particular, the module 42 is configured to discretise the propagation space Z, and for each desired discrete location across the discretised propagation space Z, apply the waveform propagation algorithm thereby to generate a hologram at that discrete location in the propagation space Z.

The module 42 may be configured to discretise the propagation space into a predetermined number of locations or zones for the purposed hereinbefore described, for example, depending on criteria such as computational efficiency, resolutions and accuracy considerations. To this end, it will be appreciated that the module 42 advantageously is configured to receive information indicative of at least the dimensions of the propagation space Z.

In a preferred example embodiment, the waveform propagation algorithm typically carries out or applies a method as described by the following waveform propagation equation (1):

$$\begin{matrix}I ( {α}^{′} , {β}^{′} ) = \frac{j}{λ} {\int }_{- ∞}^{∞} {\int }_{- ∞}^{∞} h ( x , y ) {E}_{R} ( x , y ) \frac{{e}^{( - j {kr}^{′} ) }}{r} d x d y & ( Equation 1 ) \\ {r}^{′} = \sqrt{{( x - {α}^{′} )}^{2} + {( y - {β}^{′} )}^{2} + {z}^{2}} & ( Equation 2 ) \\ k = \frac{2 π}{λ} & ( Equation 3 )\end{matrix}$$

In the forward direction, when used for hologram generation, equation 1 gives I(α′,β′) which is the complex diffraction pattern formed at the imaging/sensor plane.
   This complex diffraction pattern is then combined with the reference wave to give the Holographic intensity pattern.
   h(x,y) is then treated as the image of the object of interest
   E_{R}(x,y) is the reference wave
      r′ is the straight line distance from a point in the plane of the object to a point in the plane of the complex diffraction pattern which is used to form the hologram.
   λ is the source wavelength.
   z is the axis of propagation
   (x,y) is now the plane in which the object lies
   (α′,β′) is the plane in which the diffraction pattern, which is used to form the hologram, lies.
In the reverse direction, when used for object reconstruction, equation 1 gives I(α′,β′) which is the reconstruction of the object of interest at the location where the original object was.
   h(x,y) is then treated as the holographic intensity pattern
   E_{R}(x,y) is the reference wave
   r′ is the straight line distance from a point in the plane of the hologram to a point in the plane of the object of interest.
   λ is the source wavelength.
   z is the axis of propagation
   (x,y) is now the plane in which the hologram lies
   (α′,β′) is the plane in which the object of interest lies.

The equation (1) is used by the module 42 to generate artificial or model holographic intensity patterns or snapshots corresponding to particular discrete locations across the propagation space Z with the image received thereby as an input.

The propagation space Z in the context of determining the object descriptors will be understood to be substantially similar to the description furnished above with respect to identifying objects. In other words, the same hardware setup of the means 22 used in determining the object descriptors may ideally be substantially similar to the hardware setup used in identifying the objects, in this way, the dimensions of the propagation space Z is known by the server 12.

Regarding the selection of equation (1) for use by the module 42, it will be appreciated that the waveform propagation equation (1), in a sense, functions as a lens. It brings objects into focus. When the objects are in focus (as in a typical lens) the light waves are made to coincide at the point of focus while at other points they exist in various degrees of separation from each other. This is possible because the embedded phase information allows depth reconstruction which means that objects at different distances can be separated.

Another important point is that equation (1) describes the relationship of all the light waves at any point in the three-dimensional propagation space. If a sample of the propagating light is captured at some point in three-dimensional space, then equation (1) would allow the reconstruction of the point at another location.

In other words, the waveform propagation equation (1) firstly maintains the relationship of light waves through the propagation space Z and secondly functions as a lens (or transform on the light waves) and separates out the light waves from each other (or focuses them), these two operations are combined (and exploited) to create variations in propagation space Z.

The module 42 further comprises a training key-point extraction module 42 configured to determine descriptor key-points of interest or stable descriptor key-points for each generated holographic intensity pattern across the propagation space Z. This may be done in a conventional manner to extract key-points of interest. For example, a variety of saliency detectors may be applied over the propagation space Z. Salient points that occur across the propagation space Z are identified as points that are invariant across the propagation space Z. This particular subset will contribute to the detection or identification process only but in a stable manner.

The module 42 is then configured to use the determined descriptor key-points and information indicative of the associated discrete locations across the propagation space to generate the object descriptor associated with the object, for example, a red blood cell. This may be done by generating the descriptor subsets by associating the descriptor key-points, identified by vectors, with the respective or corresponding discrete locations in the propagation space Z in a manner as described above for each snapshot generated by the wave propagation module 42. Once a plurality of descriptor subsets are generated for a particular object across the propagation space Z, the module 42 associates and stores the same in the database 18 as the object descriptor, for use by the system 12 to identify objects irrespective of their location in the propagation space Z.

In practical applications the invention allows for an object to be advantageously identified from a single snapshot of the hologram without having to refocus and search through the holographic reconstructions to first find the object.

The server 12 may use the above principles and implement a statistical machine configured to apply a learning algorithm, for example a neural network, which could be trained to derive features automatically and to further use these to generate object descriptors (automatically) for identification without the more discrete derivation of features or set of descriptors. The system 10 may be configured to generate holograms for training the statistical machine.

In a preferred example embodiment, in addition to being propagation space Z invariant, the object descriptors can be made to be scale space invariant thereby to identify an object of interest across the propagation space as well as scale space S. Scale space invariance may be an add-on functionality of the invention.

To enable the image processor 36 to make use of scale-space theory technique, wavelets may be used as base functions—where the image information is represented by summing different pulses together. Wavelets allow for the frequency and the spatial coordinates of the image to be visualised on the same plot. In the system, information is distributed across the scale-space. Applying wavelets to the space allow us to find this information and group it together.

As the focal distance between the object and the image within the scale-space changes, the image of the object gets more blurred, giving a spatial representation of the object. By finding the stable points along the entire spatial representation, i.e. at each image point from the object, features can be extracted.

A collection of these stable points is then grouped to become a vector, which can be used for classification of objects, as a vector can be created per class of object. By collecting pieces of information across the scale-space, objects can be uniquely identified.

In one example embodiment, the memory device 18 may store a plurality of user profiles associated with users of the system 10. The user profiles may comprise information associated with the user, medical history and history associated with outputs generated by the system 10 for the user. The user profile may be accessible by the password entered by the user via the station 14. It follows that though not illustrated or described further, the user may register to use the system 10.

It will be appreciated that in system 10, the bulk of the processing takes place in the remote server 12 thereby to minimise processing required by the stations 14. However, it will be understood that if desired, most of the system 10 as hereinbefore described may be located in a handheld portable device. It follows that this may advantageously be realised by provision of the convenient and computationally efficient processing techniques and methodologies described herein.

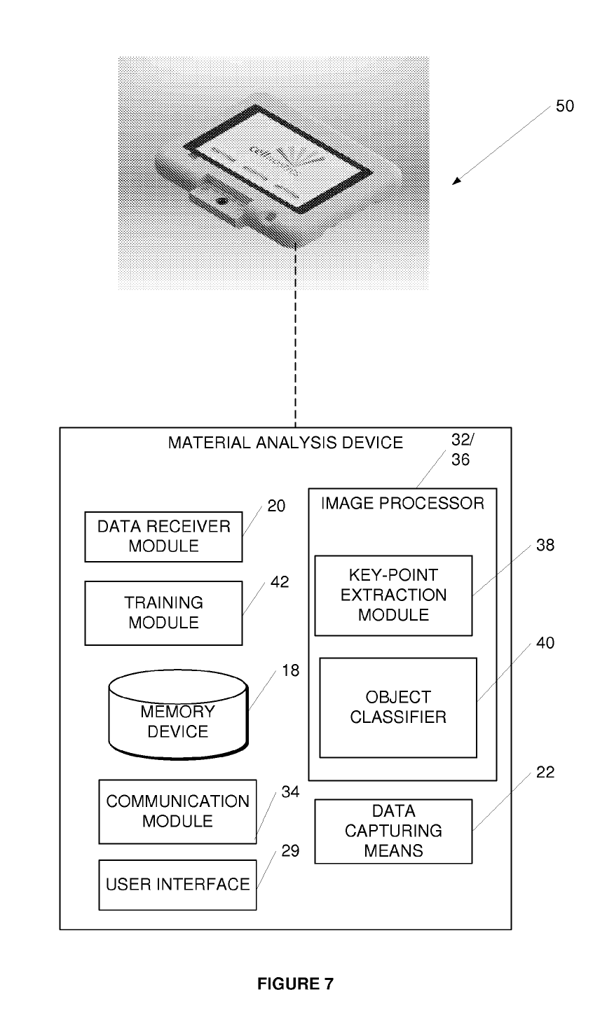

Referring now to FIG. 7 of the drawings, a material analysis device in accordance with a preferred example embodiment of the invention is generally indicated by reference numeral 50.

The device 50 is substantially similar to the station 14 and comprises all the components thereof as hereinbefore described save a few differences. In addition, the device 50 additionally comprises most of the components of the system 10, particularly the server 12, in the housing 14.1. For this reason, like parts will be indicated by like reference numerals and it follows that the descriptions of the various components as provided above apply to FIG. 7, as the case may be and as practicable, for example, it will be understood that none of the components of the device 50 are distributed across networks, as was the case for the server 12, but optionally communicatively hardwired together and contained in a single rugged and robust portable unit.

It will be noted that the processor 32 comprises the more powerful image processor 36 as hereinbefore described. It follows that the device 50 is much more computationally dynamic than the station 14 as hereinbefore described. In the device 50, the data receiver module 20 is advantageously hardwired to the data capture device 22 to receive captured holographic intensity therefrom. In one example embodiment, the receiver module 20 may be in data communication with the image sensor 30.

The operation of the image processor 36 as hereinbefore described advantageously allows processing and analysis of the holographic intensity pattern in a much more convenient and faster manner as compared to conventional methods which are computationally expensive.

Also, it will be noted that user control inputs received via the user interface 29 are typically handled by the image processor 36 which in turn process output data and provide the same for display via the user interface, for example, the touch-screen interface.

Other operations of the device 50 are substantially similar as hereinbefore described with reference to the system 10. It will be noted that the device 50 need not operate in a vacuum and may communicate via the module 34 to a server 12 storing patient profiles, etc. as the case may be.

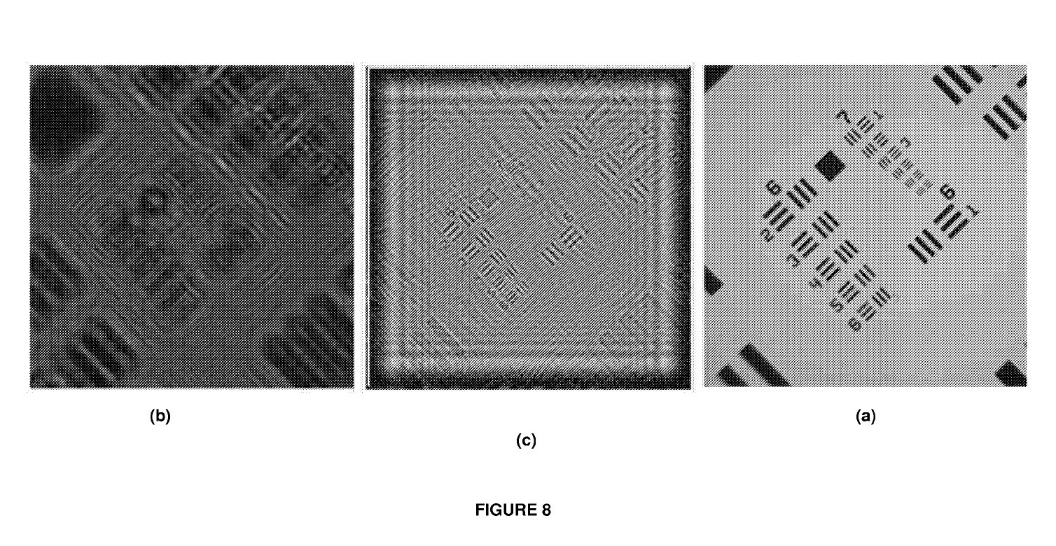

Turning now to FIGS. 8 to 11 of the drawings where example images generated by a in-line holographic microscope arrangement similar to one hereinbefore described is illustrated for completeness.

FIG. 8 (a) shows the digital hologram of the central area of a positive 1951 United States Air Force (USAF) Wheel Pattern Test Target slide (R3L1S4P, Thorlabs), recorded by a CMOS sensor on a digital in-line holography microscope platform.

The digital hologram generated was then used as an input to an image reconstruction algorithm, similar to the one applied by the system 10/device 50. The algorithm first performs pre-processing of the hologram image by means of a Laplacian filter to enhance the contrast of the hologram. The reconstructed USAF slide image is shown in FIG. 8 (b). FIG. 8 (c) shows an image of the USAF slide as captured using a CMOS sensor connected to a conventional bright field microscope with approximately 400× magnification.

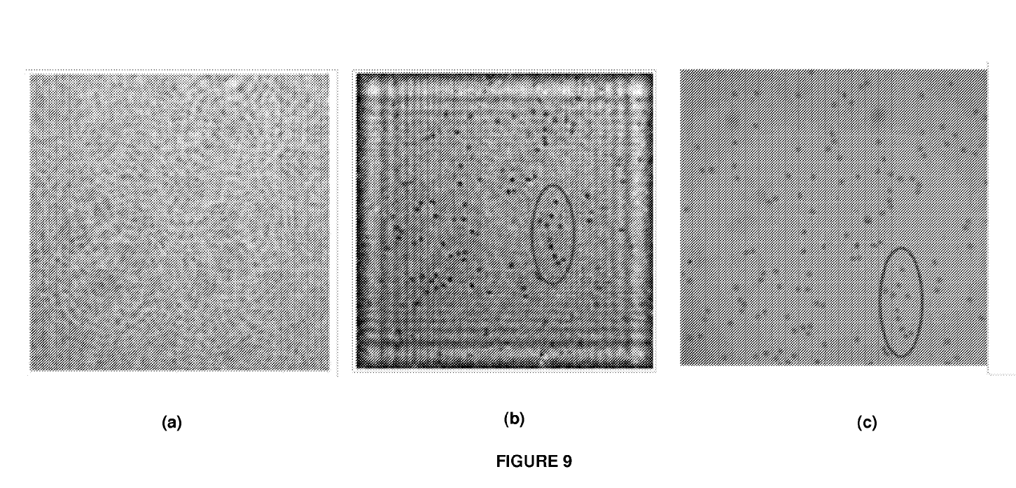

To test the abilities of the digital in-line holography microscope platform further, blood smear slides were imaged. A hologram of a small area of a blood film slide that was obtained using a blue laser diode is shown in FIG. 9 (a). The corresponding reconstructed image is shown in FIG. 9 (b), with a comparison to an image of the same area of the blood film obtained using a conventional bright field microscope with 400× magnification. The circled areas in FIGS. 9 (b) and (c) assist in highlighting corresponding areas in the two images.

The blue light source provided clearer results for imaging red blood cells, which are more prevalent than white blood cells in a blood film. This suggests that information from different light sources could be combined for optimal image reconstruction results and will be investigated further.

In some example embodiments, optimization of the digital holographic microscope arrangement, by varying different light sources, intensities, light source aperture sizes, and distances between the light source 24 and the sample and between the sample and the image sensor 30, causes variations in the generated holographic intensity data captured.

In one example embodiment, the following parameters where determined to be optimal:
Red laser diode light source (635 nm wavelength) 24
30 μm illumination aperture 26.1 at the light source 24
distance of 20 cm between the source and the sample holder 28
distance of 2 mm between the sample and the image sensor 30

For holograms captured by the means 22 under the above conditions, optimal image reconstructions were found at the following parameters set in the image reconstruction algorithm:
resolution of image (res)=320
Laplacian filter scale factor (lap)=1.4
distance between the sample holder and the image sensor 30=2380 to 2400 for red blood cells (RBCs) to be most clearly in focus
distance between the sample and the image sensor 30=2520 to 2550 for white blood cells (WBCs) to be most clearly in focus

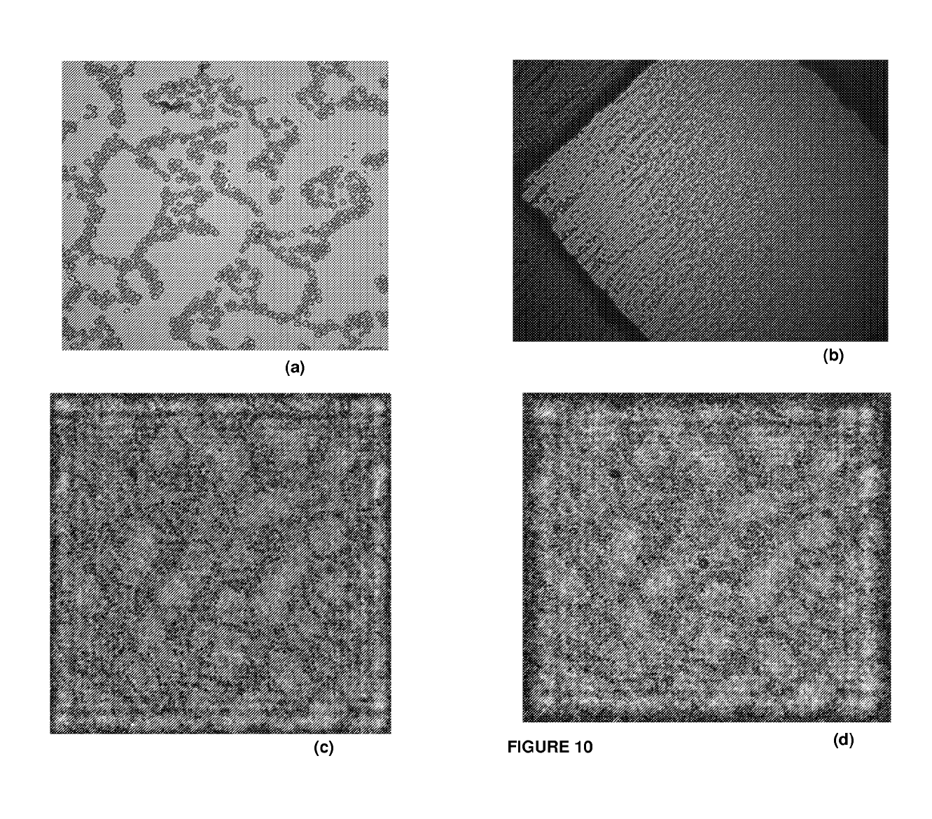

The optimized microscopy arrangement and reconstruction parameters were used for the implementation of the first integrated system. An example of the results obtained using the optimized arrangement is shown in FIG. 10.

A bright field microscope image of a small section of a standard blood obtained using the experimental platform is shown in FIG. 10 (a). The corresponding hologram over the entire field-of-view of the image sensor 30 is shown in (b), with the small section of interest that corresponds to the microscope image positioned in the centre of the hologram. The small sub-section of the centre of the hologram (approximately 300×300 pixels in size) is then analysed and the image is reconstructed. Image reconstruction for RBCs to be in focus is shown in (c), while the image reconstruction for WBCs to stand out and be in focus is shown in (d).

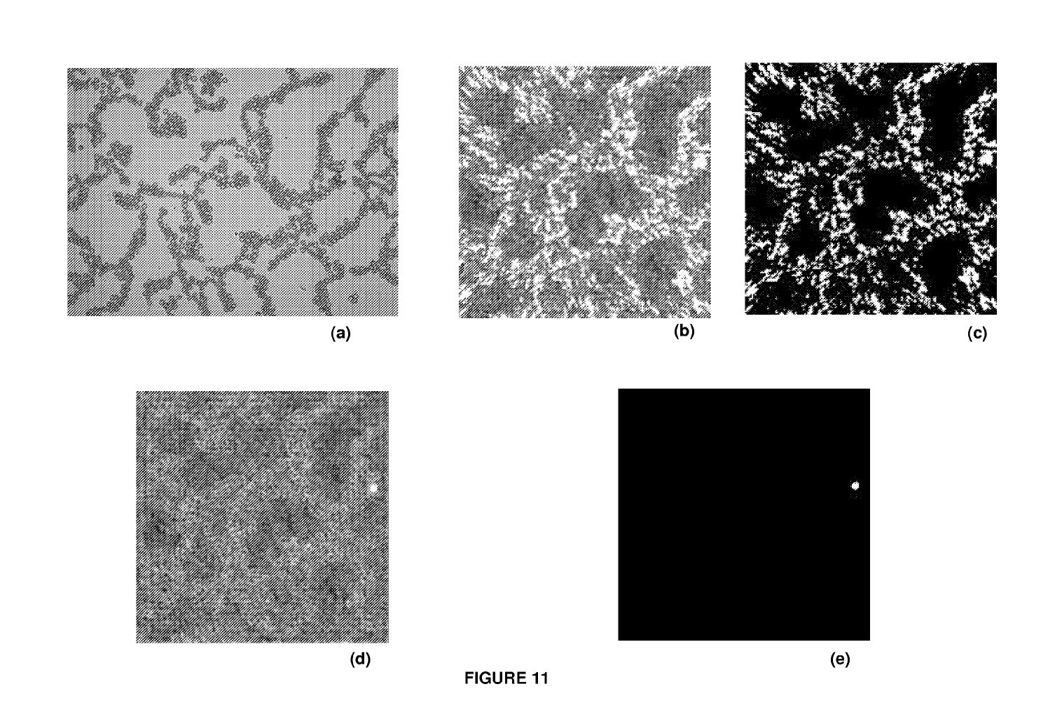

An example of the analysis results generated by the system 10/device 50 using holograms captured by the means 22 is shown in FIGS. 11. It can be seen that generally the WBC count is correctly calculated and an estimate of the RBCs is returned, finding all the cells in the correct locations.

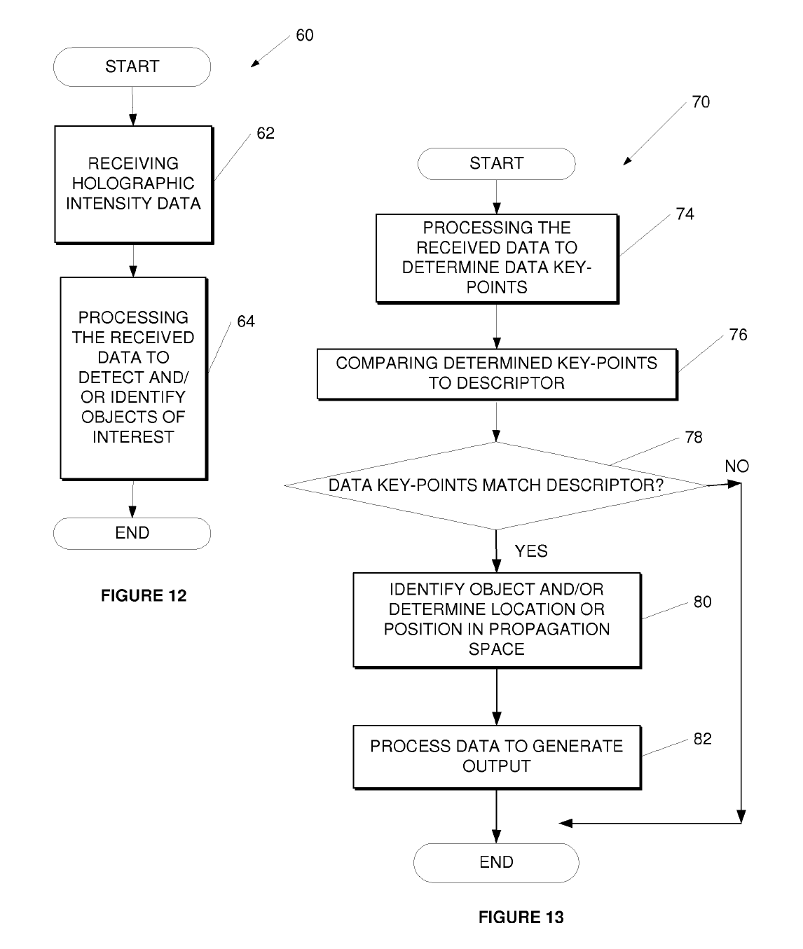

Example embodiments will now be further described in use with reference to FIGS. 12 and 13. The example methods shown in FIGS. 12 and 13 are described with reference to FIGS. 1 to 11, although it is to be appreciated that the example methods may be applicable to other systems and devices (not illustrated) as well.

In FIG. 12, a high level flow diagram of a method in accordance with an example embodiment is generally indicated by reference numeral 60. The method 60 may be described with reference to an example embodiment whereby a user using a device 50 in accordance with the invention desires to analyse a sample of blood, for example, to determine a blood count of white blood cells. Embodiments with reference to operation of the system 10 may be inferred from the explanation which follows.

The user introduces the sample of blood on a sample holder 28 and places the same on the tray of the flap 14.3 (in the first condition) of the housing 14.1 of the device 40. The user operates the flap 14.3 to introduce the sample in the sample holder to the chamber 14.1 of the housing 14. The user then operates the user interlace 29 by way of the GUI to instruct the device 50 to capture an image, particularly holographic intensity data or hologram, wherein the data capture means 22 is operated by the device 50, in response to receiving a suitable instruction from the user interface 29, to capture the hologram associated with the blood sample.

The method 60 therefore comprises receiving, at block 62, the captured hologram from the means 22 via the receiver module 20 in hardwired data communication therewith. The hologram being associated with a particular location in propagation space Z associated with the device 22.

In response to receiving the hologram, the method 60 comprises processing, at block 64 by way of the image processor 36, the received hologram thereby at least to detect or identify one or more objects of interest, e.g., white blood cells in the sample of blood from the associated hologram. The processor 36 may count the number of white blood cells successfully detected or identified from the received hologram and generate output data comprising at least a white blood cell count associated with the sample of blood.

This output data may typically be displayed via the user interface 29, for example, in real-time, or near real-time to the user. The processor 36 may reconstruct an image from the hologram in a conventional manner and may output the same, and optionally annotate the same with output data determined.

In FIG. 13, a high level flow diagram of a method in accordance with an example embodiment is generally indicated by reference numeral 70. The method 70 is typically related to the method of FIG. 12, particularly step 64 of FIG. 13.

The method 70 comprises processing, at block 74 by way of the processor 36, received holographic intensity data to determine data key-points of a potential object of interest, i.e., a white blood cell in the received holographic intensity image. In some example embodiments, the determination of the data key-points may entail the extraction of external points from a difference of Gaussians and the generation of a vector for each determined data key-point of interest by the module 38, for example.

The method 70 then comprises comparing, at block 76 & 78, for example, by way of the object classifier 40, the determined data key-points to at least one pre-determined object descriptor stored in the memory device 18. The method 70 comprises comparing each determined data key-point, particularly information associated therewith, with descriptor key-points of propagation space invariant descriptors as described above in order to determine a match wherein the descriptor are propagation space invariant and optionally scale space invariant. It will be noted that the method 70 may comprise the steps (not shown) of determining the object descriptors by operating the training module 42 to operate in a manner as hereinbefore described.

If the comparison step 76/78 results in a match, then the method 70 correspondingly identifies, at block 80 by way of the module 40, that the object associated with the determined data key-points is a white blood cell as the matching descriptor key-point of the object descriptor is typically associated with the object which in this case is a white blood cell.

The method 70 may be repeated for each data key-point of interest in the received holographic image.

The method 70 may further comprise, at block 82, processing determined data to produce output data, for example, for classifying the objects by counting detected or identified objects, generating reconstructed images from the received holograms, and the like.

Though described in detail above, it may be worth re-iterating in other words that the feature extraction process for more specific object identification utilises the Fresnel-Kirchoff transform as the mechanism to represent information about an object of interest across a continuous space, which is the space defined by the axis of propagation.

The isolation of stable points is carried out across this space, to allow for a collection of stable points to be used as a vector in a classifier. This then enables individual and distinct objects of interests to be identified by means of a unique signature, providing a novel method of feature extraction.

To find the stable points, a number of different methods can be employed. These techniques may include, but are not limited to, location of the local maxima and minima positions or stationary points, Fourier descriptors, moment invariance, and principal component analysis. The stable points extracted that are common to the information across the whole space would then be indicative of points that would be stable across the whole space. By combining these common stable points, together they form a stable signature that identifies the object of interest across the entire propagation space.

The collection of stable points obtained can be used as a vector in a classifier, examples of which include but are not limited to neural networks. This allows the feature extraction process to perform an identification of an object of interest from information that is measured and captured at only one point along the axis of propagation, but using information extracted from the entire space along the axis of propagation.

The invention thus allows for a stable set of features to be extracted to be used for the classification of objects of interest. In order to do this, the process finds stable features across the entire transformation space, encompassing a much broader scope that existing techniques for obtaining hologram signatures, where only one point or a single snapshot along the axis of propagation is used. By using a broader space to extract hologram signatures, the invention provides a more robust identifier than just using a single snapshot, with a higher tolerance.

The feature extraction process of the invention is also advantageous for any type of depth measurement to be successfully achieved, as the process is independent of where the object lies along the axis of propagation. Thus, the objects of interest could lie at different depths or layers within a volume, but individual signatures could still be extracted for every object, regardless of its position within the volume. For analysis of samples with multi-layers, the invention thus provides an improved and more robust identifier.

The information extraction process of the invention can further be enhanced by applying multi-spectral techniques, by changing the light source in the optical set-up. Different types of objects create different spectra under changing wavelengths of light sources. This can be used as an additional classification mechanism. For the current system, only a red light source has been used, but a variety of other light sources with different wavelengths can be explored. A signature for an object under different wavelengths can be formulated, and by combining the signatures at different wavelengths, a combined, stronger signature can be obtained.

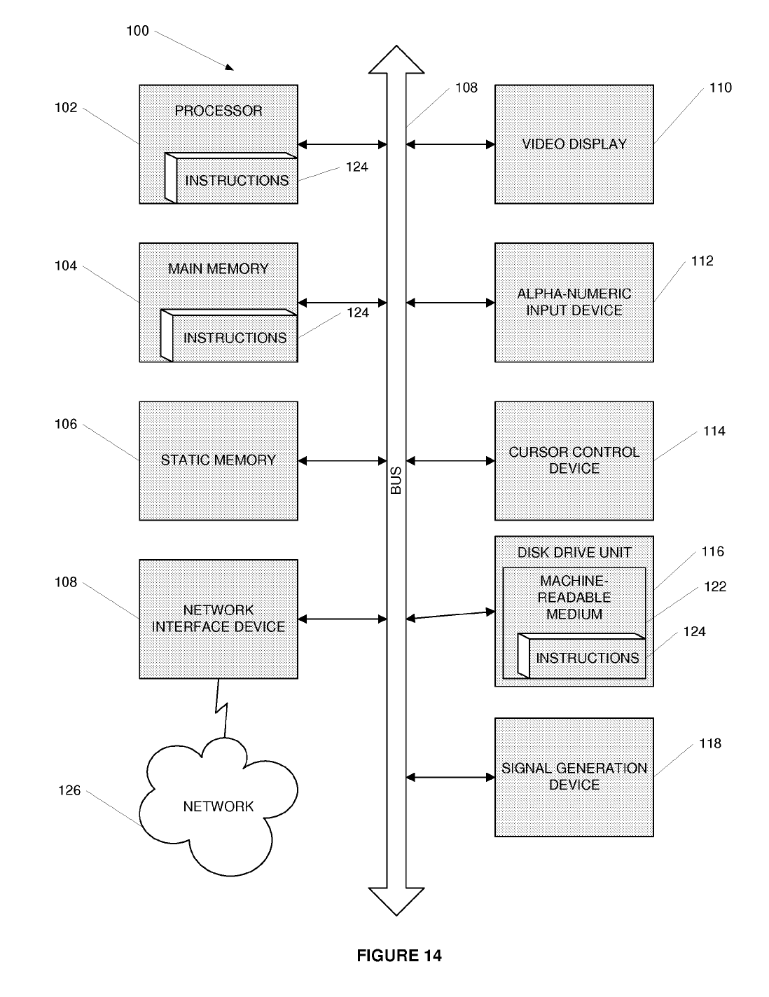

FIG. 14 shows a diagrammatic representation of machine in the example of a computer system 100 within which a set of instructions, for causing the machine to perform any one or more of the methodologies discussed herein, may be executed. In other example embodiments, the machine operates as a standalone device or may be connected (e.g., networked) to other machines. In a networked example embodiment, the machine may operate in the capacity of a server or a client machine in server-client network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated for convenience, the term “machine” shall also be taken to include any collection of machines that individually or jointly execute a set (or multiple sets) of instructions to perform any one or more of the methodologies discussed herein.

In any event, the example computer system 100 includes a processor 102 (e.g., a central processing unit (CPU), a graphics processing unit (GPU) or both), a main memory 104 and a static memory 106, which communicate with each other via a bus 108. The computer system 100 may further include a video display unit 110 (e.g., a liquid crystal display (LCD) or a cathode ray tube (CRT)). The computer system 100 also includes an alphanumeric input device 112 (e.g., a keyboard), a user interface (UI) navigation device 114 (e.g., a mouse, or touchpad), a disk drive unit 116, a signal generation device 118 (e.g., a speaker) and a network interface device 120.

The disk drive unit 16 includes a machine-readable medium 122 storing one or more sets of instructions and data structures (e.g., software 124) embodying or utilised by any one or more of the methodologies or functions described herein. The software 124 may also reside, completely or at least partially, within the main memory 104 and/or within the processor 102 during execution thereof by the computer system 100, the main memory 104 and the processor 102 also constituting machine-readable media.

The software 124 may further be transmitted or received over a network 126 via the network interface device 120 utilising any one of a number of well-known transfer protocols (e.g., HTTP).

Although the machine-readable medium 122 is shown in an example embodiment to be a single medium, the term “machine-readable medium” may refer to a single medium or multiple media (e.g., a centralized or distributed database, and/or associated caches and servers) that store the one or more sets of instructions. The term “machine-readable medium” may also be taken to include any medium that is capable of storing, encoding or carrying a set of instructions for execution by the machine and that cause the machine to perform any one or more of the methodologies of the present invention, or that is capable of storing, encoding or carrying data structures utilised by or associated with such a set of instructions. The term “machine-readable medium” may accordingly be taken to include, but not be limited to, solid-state memories, optical and magnetic media, and carrier wave signals.

The present invention provides a convenient manner for processing and analysing material, particularly samples thereof. Conventional digital holography systems (particularly for microscopy applications) have focussed on optimizing the optical and physical set-ups of the system, in order to obtain holograms that yield optimal reconstructed and in focus images. These optical systems can become bulky, expensive and complex, and are very sensitive to external/environmental factors.

The invention provides an integrated, self-contained and connected system utilising a simple physical set-up, made possible by computationally efficient information extraction techniques and signal processing methodologies thus allowing for the device to be compact and rugged, ideally suited as a Point Of Care (POC) device. The system is a self-contained mobile POC device, that contains the sensor/measurement device and also contains the interface to the system and optionally to the server, where the computationally intensive analysis/processing occurs and patient data is stored. A patient database is implemented, allowing for patient medical history and results files to be stored and accessible from anywhere in the world at any time. The target of this system is towards the application area of medical clinical environments for the purpose of speeding up analysis and diagnosis. The integrated system for the current application speeds up blood analysis from the time of measurement to the time that the report is generated. This can be applied to any analysis or diagnostic application where rapid analysis and diagnosis times are of importance.

In addition, the invention provides convenient methods to extract maximal information for object identification. This includes a novel feature extraction process for object identification. This latter process makes use of the Fresnel-Kirchoff transform as the mechanism to allow for extraction of information across the entire propagation space. Features can be extracted to allow for unique signatures to be created for each different object under investigation. This information can then be used to implement a novel classification method for identifying objects without needing to first obtain a reconstructed image with high visual quality and high resolution for object identification.

Instead of focussing on refining the physical set-up to obtain high quality reconstructed images, the invention focuses on extracting maximal information from the hologram. Image reconstruction quality and thus physical system set-up is not the focus, rather the information extraction using the available information is of primary concern.

As the invention uses simple hardware, without complex optical set-ups, but still allows the extraction of sufficient information of interest, it introduces a fresh approach to the successful and robust implementation of digital holography-based systems.

1. A method of analysing material, the method comprising:
receiving holographic intensity data comprising at least a holographic intensity pattern associated with a sample of a material of interest, wherein the holographic intensity data is captured by a holographic microscope arrangement; and
processing the received holographic intensity data at least to perform one or both steps of detecting and identifying at least one object of interest in the sample.

2-23. (canceled)

24. The method as claimed in claim 1, wherein the step of processing the received holographic intensity data comprises:
determining one or more data key-points from the received holographic intensity data, wherein the received holographic intensity data is associated with a discrete location in a propagation space, and wherein the propagation space comprises a three-dimensional space over which illumination from an illumination source propagates to facilitate capturing of the holographic intensity data; and
comparing the one or more determined data key-points to at least one pre-determined object descriptor associated with an object to determine a match, thereby facilitating one or both steps of detecting and identifying at least one object of interest in the sample, wherein the at least one object descriptor is propagation space invariant.

25. The method as claimed in claim 24, the method further comprising providing a plurality of object descriptors corresponding to a plurality of objects, wherein each object descriptor comprises a plurality of descriptor subsets associated with a plurality of desired discrete locations in the propagation space respectively, and wherein each descriptor subset comprises one or more descriptor key-points.

26. The method as claimed in claim 24, wherein the method comprises the prior steps of determining the object descriptors, which steps comprising, for each object:
receiving an image of the object;
applying a waveform propagation algorithm to the received image for a plurality of discrete locations across the propagation space thereby to generate a plurality of holographic intensity patterns corresponding to the discrete locations across the propagation space;
determining descriptor key-points for each generated holographic intensity pattern across the propagation space; and
using the determined descriptor key-points and information indicative of the associated discrete locations across the propagation space to generate the object descriptor associated with the object.

27. A method as claimed in claim 1, wherein the method comprises receiving holographic intensity data in either a hardwired fashion from the holographic microscope arrangement or wirelessly from a plurality of geographically distributed analysis stations each comprising holographic microscope arrangements.

28. The method as claimed in claim 1, wherein the method comprises controlling the holographic microscope arrangement to generate holographic data of the sample, wherein the holographic data comprises at least a holographic intensity pattern associated with the sample.

29. The method as claimed in claim 1, wherein the method comprises:
generating output data associated with one or both of the detection and identification operations; and
transmitting the output data in a hardwired or a wireless fashion to a user interface module at least for output thereby.

30. The method as claimed in claim 29, wherein the method comprises:
classifying detected or identified objects of interest by determining a sum of similar objects of interest;
generating an image of the sample by reconstructing the received holographic intensity data;
generating output data comprising one or both of the determined sum and the generated image of the sample; and
transmitting the output data in a hardwired or a wireless fashion to a user interface module for output thereby.

31. A material analysis system comprising:
a memory device storing data;
a data receiver module, which is in data communication with a holographic microscope arrangement, wherein said data receiver module is configured to receive holographic intensity data comprising at least a holographic intensity pattern associated with the sample of the material of interest captured by the holographic microscope arrangement; and
an image processor configured to process the received holographic intensity data at least to perform one or both operations of detecting and identifying at least one object of interest in the sample.

32. The material analysis system as claimed in claim 31, wherein the image processor comprises:
a key-point extraction module configured to determine one or more data key-points from the received holographic intensity data, wherein the holographic intensity data is associated with a discrete location in a propagation space, and wherein the propagation space comprises a three-dimensional space over which illumination from an illumination source propagates to facilitate capturing of the holographic intensity data; and
an object classifier configured to compare the determined data key-points to at least one pre-determined object descriptor associated with an object to determine a match thereby facilitating one or both steps of detecting and identifying at least one object of interest in the sample, wherein the object descriptor is propagation space invariant.

33. The material analysis system as claimed in claim 32, wherein the memory device stores a plurality of object descriptors, wherein each object descriptor is associated with an object, and wherein each object descriptor comprises a plurality of descriptor subsets associated with a plurality of desired discrete locations in the propagation space respectively, wherein each descriptor subset comprises one or more descriptor key-points.

34. The material analysis system as claimed in claim 32, wherein the material analysis system comprises a training module configured to determine the object descriptors, wherein the training module is configured, for each object, to:
receive an image of the object;
apply a waveform propagation algorithm to the received image for a plurality of discrete locations across the propagation space thereby to generate a plurality of holographic intensity patterns corresponding to the discrete locations across the propagation space;
determine descriptor key-points for each generated holographic intensity pattern across the propagation space; and
use the determined descriptor key-points and information indicative of the associated discrete locations across the propagation space to generate the object descriptor associated with the object.

35. The material analysis system as claimed in claim 32, wherein the system comprises the holographic microscope arrangement or a plurality of geographically distributed analysis stations each comprising a holographic microscope arrangement, wherein each holographic microscope arrangement comprises the said illumination source, wherein the illumination source is configured to generate illumination, and an image sensor configured to generate holographic intensity data in response to the generated illumination incident thereon from the illumination source, in use, wherein the propagation space comprises at least part of the three-dimensional space between the illumination source and the image sensor.

36. The material analysis system as claimed in claim 35, wherein the holographic microscope arrangement further comprises:
a spatial filter located at a predetermined distance from the illumination source, the spatial filter comprising at least one illumination aperture for passage of illumination from the illumination source therethrough; and
a sample holder removably locatable at a predetermined distance from the spatial filter, the sample holder being configured to hold the sample of material of interest, wherein the image sensor is spaced from the sample holder such that, in use, illumination from the illumination source propagates from the illumination source through the illumination aperture, through the sample holder holding the sample of the material of interest, and impinges onto the image sensor which, in response to the illumination incident thereon, generates the holographic intensity data of the sample of the material of interest; wherein the propagation space comprises the three-dimensional space over which illumination from the illumination source, or illumination propagating from one or both of the illumination aperture and sample holder, propagates to reach the image sensor thereby to form the holographic intensity data.

37. The material analysis system as claimed in claim 31, wherein the system is a biomaterial analysis system for analysing a sample of biomaterial associated with a human user, the system therefore comprising a user interaction module configured to generate a user profile for at least one user of the system in the memory device, the user profile storing generated output data associated with a particular user.

38. A material analysis device comprising:
a housing configured removably to receive an sample holder carrying a sample of a material of interest, in use;
a holographic microscope arrangement located in the housing and configured to capture a holographic intensity pattern of the sample of the material of interest;
a memory device storing data;
an image processor configured to process the captured holographic intensity data at least to perform one or both operations of detecting and identifying at least one object of interest in the sample thereby to generate output data associated with said operations; and
a user interface configured to receive user input and to output information comprising at least output data generated by the image processor.

39. The material analysis device as claimed in claim 38, wherein the image processor comprises:
a key-point extraction module configured to determine one or more data key-points from the received holographic intensity data, wherein the holographic intensity data is associated with a discrete location in a propagation space, and wherein the propagation space comprises a three-dimensional space over which illumination from an illumination source propagates to facilitate capturing of the holographic intensity data; and
an object classifier configured to compare the determined data key-points to at least one pre-determined object descriptor, stored in the memory device, associated with an object to determine a match thereby facilitating one or both steps of detecting and identifying at least one object of interest in the sample, wherein the object descriptor is propagation space invariant and comprises a plurality of descriptor subsets associated with a plurality of desired discrete locations in the propagation space respectively, and wherein each descriptor subset comprises one or more descriptor key-points.

40. The material analysis device as claimed in claim 38, wherein the holographic microscope arrangement comprises:
the said illumination source configured to generate illumination;
a spatial filter located at a predetermined distance from the illumination source, the spatial filter comprising at least one illumination aperture for passage of illumination from the illumination source therethrough; wherein the sample holder is removably locatable at a predetermined distance from the spatial filter; and
an image sensor spaced from the sample holder, the image sensor being configured to generate at least a digital holographic intensity pattern of the material of interest in the sample holder in response to generated illumination incident thereon, in use, wherein the propagation space comprises a space over which illumination from illumination source, or propagating from one or both of the illumination aperture and sample holder propagates, to reach the image sensor thereby to form the holographic intensity data.

41. The material analysis device as claimed in claim 38, the device comprising:
a communication module configured to receive data and transmit data wirelessly from the device; and a user interaction module configured to generate a user profile for at least one user of the device in the memory device, wherein the user profile stores generated output data associated with a particular user of the device.

42. A non-transitory computer readable storage medium comprising a set of instructions, which when executed by a computing device causes the same to perform a method comprising:
receiving holographic intensity data comprising at least a holographic intensity pattern associated with a sample of a material of interest, wherein the holographic intensity data is captured by a holographic microscope arrangement; and
processing the received holographic intensity data at least to perform one or both steps of detecting and identifying at least one object of interest in the sample.
